(19) United States
(12) Patent Application Publication
(10) Pub. No.: US 2016/0302972 A1
(43) Pub. Date: Oct. 20, 2016

(54) Nasal Air Flow Device

(71) Applicant: Exercore LLC, Minneapolis, MN (US)

(72) Inventors: Kevin Connelly, Chaska, MN (US); Derek Lidbom, Plymouth, MN (US)

(73) Assignee: EXERCORE LLC, Minneapolis, MN (US)

(21) Appl. No.: 14/687,669

(22) Filed: Apr. 15, 2015

(51) Int. Cl. A61F 11/00; A61M 13/00

(52) U.S. Cl. CPC A61F 11/00; A61M 13/003; A61M 2210/0668

(57) ABSTRACT

An apparatus for equalizing pressure and exercising the Eustachian tubes includes a hand held air source for providing a flow of air into a nostril while the other nostril is shut. Swallowing during the intake of air in the nostril helps to open the Eustachian tubes and routine use on a daily can prevent problems associated with flying, scuba diving, colds and allergies. A simpler and less expensive air pressure limiting device limits the air pressure perceived at the nose by way of a unique air tube configured with simple side openings and screws adjusted when manufactured to preset the maximum air pressure.

### CROSS-REFERENCE TO RELATED APPLICATIONS

None.

### STATEMENT REGARDING FEDERALLY SPONSORED RESEARCH

Not Applicable

### BACKGROUND OF THE INVENTION

1. Field of the Invention

The present invention relates to a nasal air flow device and, more particularly, to a device for exercising the Eustachian tubes.

Eustachian tubes normally open with swallowing and yawning exercises. When the Eustachian tubes fail to open, ear problems may occur. A person may attempt to exercise their Eustachian tubes by yawning, chewing, swallowing and pinching their nose and blowing out (called the Valsalva maneuver). However, the disclosed techniques do not always work.

2. Description of the Related Art

U.S. Pat. No. 5,419,762 to Arick et al. which issued May 30, 1995 discloses a device for equalizing the middle ear pressure. U.S. Pat. No. 5,885,242 of Arick et al. which issued Mar. 23, 1999 is a Continuation from the '762 patent. U.S. Pat. No. 7,285,123 to Silman et al. which issued Oct. 23, 2007 concerns a flexible tube inserted in a nostril and a pacifier like device inserted into the mouth that has an air bladder such that swallowing squeezes the pacifier and forces air into the nostril.

U.S. Pat. No. 7,484,531 to Hoogenakker et al, which issued on Feb. 3, 2009, concerns a middle ear pressure equalizing device that includes a plurality of exhaust ports and corresponding check valves each set to open at different predetermined pressures. It is obviously fairly complicated leading to a more expensive device that may be justified in a doctor's office but not for the usual home individual. The disclosures of U.S. Pat. Nos. 5,419,762; 5,885,242, 7,285,123 and 7,484,531 are incorporated herein by reference.

As can be seen, there is a need for a device to aid in exercising the Eustachian tubes and relieving middle ear pressure safely at a reasonable cost.

### BRIEF SUMMARY OF THE INVENTION

The invention provides a nasal airflow device that includes a body in which air may be propelled out into a nose piece that may be placed against a nostril while the other nostril is closed. The Eustachian tubes may be exercised and kept freely open by repeated use of the device. Air leading to the nose piece is easily regulated by the addition of a tube in which at least one side opening is formed such that over-pressure is prevented by allowing air to escape through alternate paths in the event that the nasal cavity is too clogged to allow ready passage of air.

The one or more side openings effectively function in a manner similar to a soaker garden hose in which appreciable flow is allowed through the end but the pressure is reduced by the side openings. Further pressure controls may include a simple screw mechanism through one or more or the side openings to allow the manufacturer to dial in the exact pressure to the nostril desired by opening or closing the side opening as needed.

While the figures focus on a device having an electrical motor to develop the needed air pressure, a simple air bladder may be used that has the inventive pressure control of side openings and/or screw controls. In a non-electrical device, it would be very inexpensive and could readily be carried on trips such as in an airplane where altitude changes cause ear pain without careful regulation of pressure. Since most people do not know Valsalva techniques or other procedures to equalize ear pressure to ambient air, airplane travel is often dreaded due to possible ear pain.

### BRIEF DESCRIPTION OF THE SEVERAL VIEWS OF THE DRAWING(S)

FIG. 1 is a side view of the device with the nosepiece in place;

FIG. 2 is a side view of the device of FIG. 1 with the nosepiece taken off;

FIG. 3 is a view similar to FIG. 2 in which parts have been removed to show the underlying air tube;

FIG. 4 is a cross-sectional view of the device lengthwise showing the interior components;

FIG. 5 is a view of the cross-section of FIG. 4 rotated;

FIG. 6 is a perspective view of the nosepiece;

FIG. 7 is a perspective view of the inside of the nosepiece;

FIG. 8 is a perspective enlarged view of the region encircled in FIG. 3;

FIG. 9 is a cross-section from FIG. 8;

FIG. 10 is an alternate embodiment as in FIG. 8 having a plurality of side openings; and

FIG. 11 is the device of FIG. 10 in which at least one side opening may be occluded with a screw.

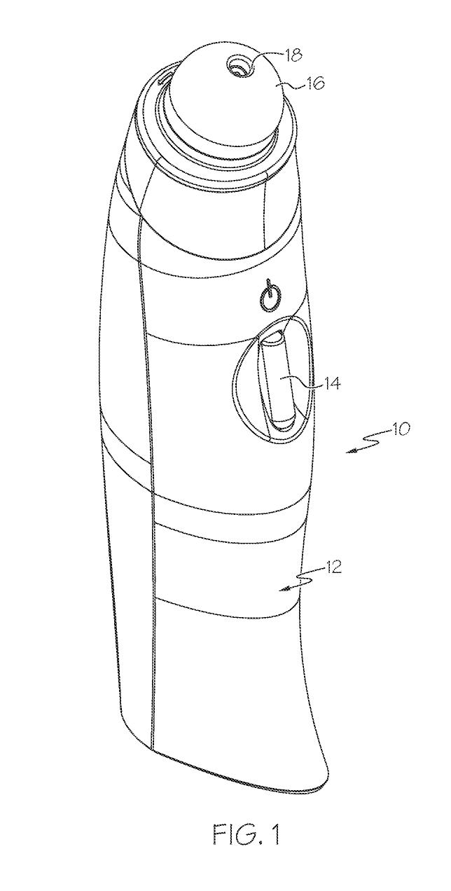

### DETAILED DESCRIPTION OF THE INVENTION

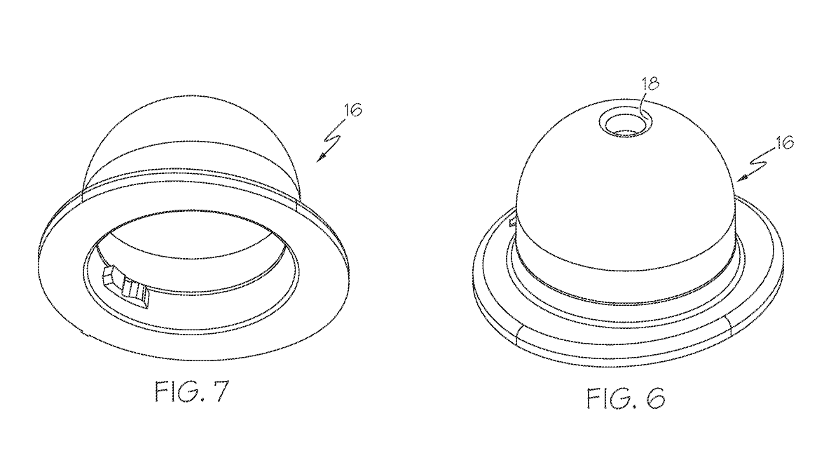

The nasal air flow device 10 of the invention as shown in the drawings is depicted as a standard hand-held, battery powered device in which a body 12 encases the components that generate air flow. A soft nosepiece is detachably mounted to an upper end 20 of the body 12 such that it may be readily removed for cleaning and disinfection before being reused. The soft nosepiece 16 includes an air exit opening 18 through which air passes. The nosepiece 16 is shown separately in FIGS. 6 and 7. A switch 14 on the body 12 is positioned to let the user readily control airflow when they position the nosepiece 16 against a nostril for use.

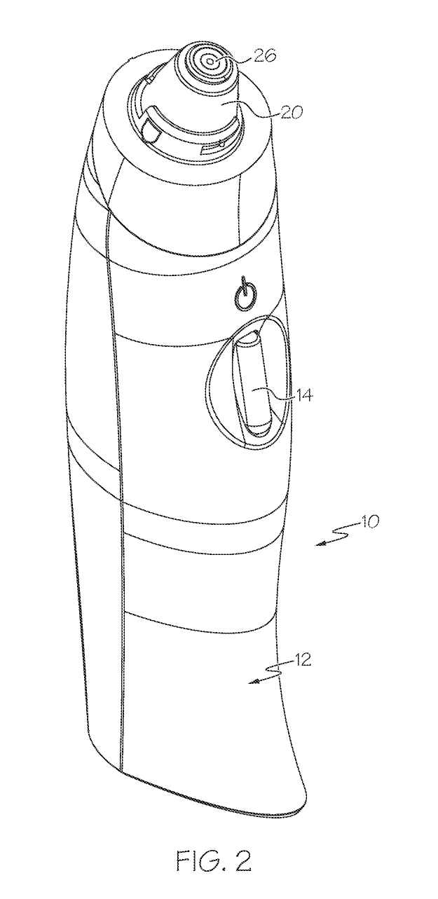

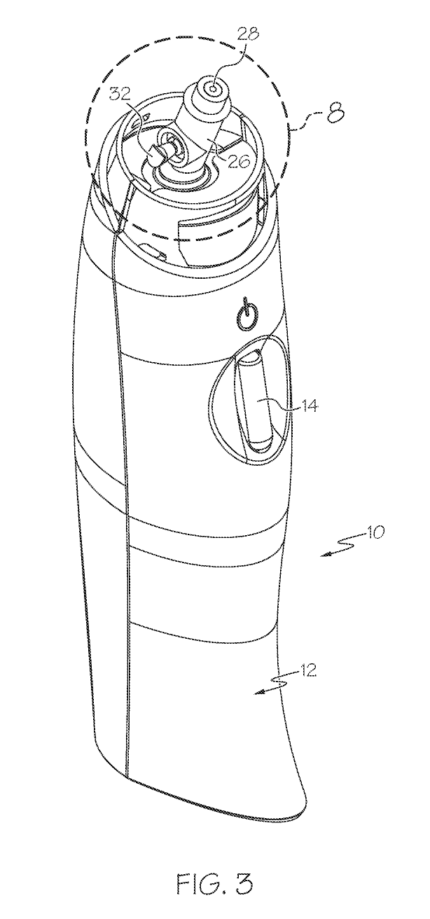

FIG. 2 shows the nosepiece 16 removed for cleaning. FIG. 3 shows the upper end 20 of the body 12 removed to show the air tube 26 which directs air under pressure out toward the air tube outlet 28. Air under pressure passes through air tube outlet 28 and into the nosepiece where it exits through the nosepiece air exit opening 18.

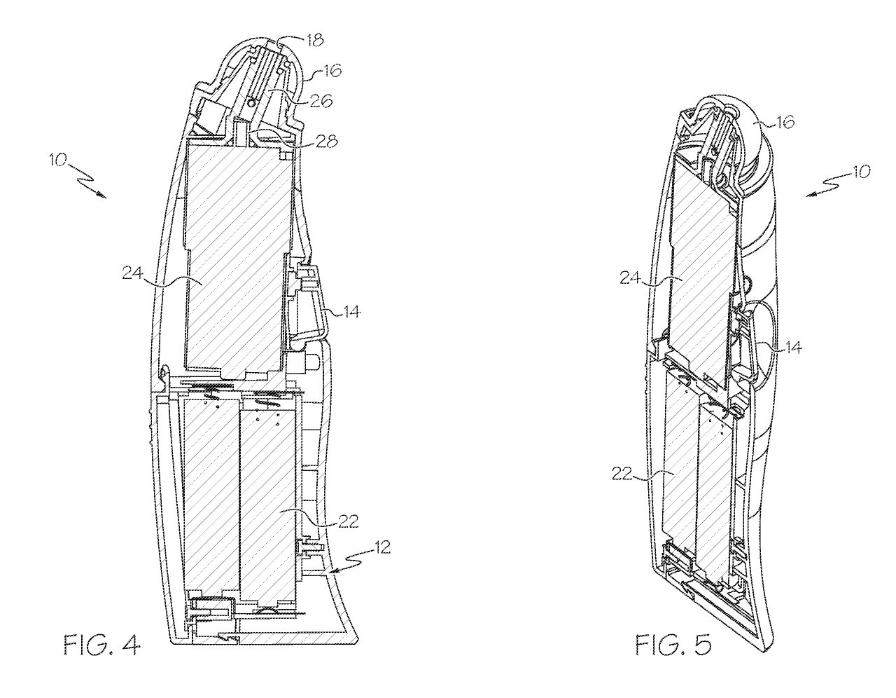

Air under pressure to air tube 26 may be supplied from a motor 24 powered by batteries 22. The motor 24 is connected to a switch 14 to turn on and off. The air generating motor 24 includes and air outlet 28 to which air tube 26 is attached. FIGS. 4 and 5 show a typical arrangement wherein the body 12 includes air generating motor 24, batteries 22 and switch 14.

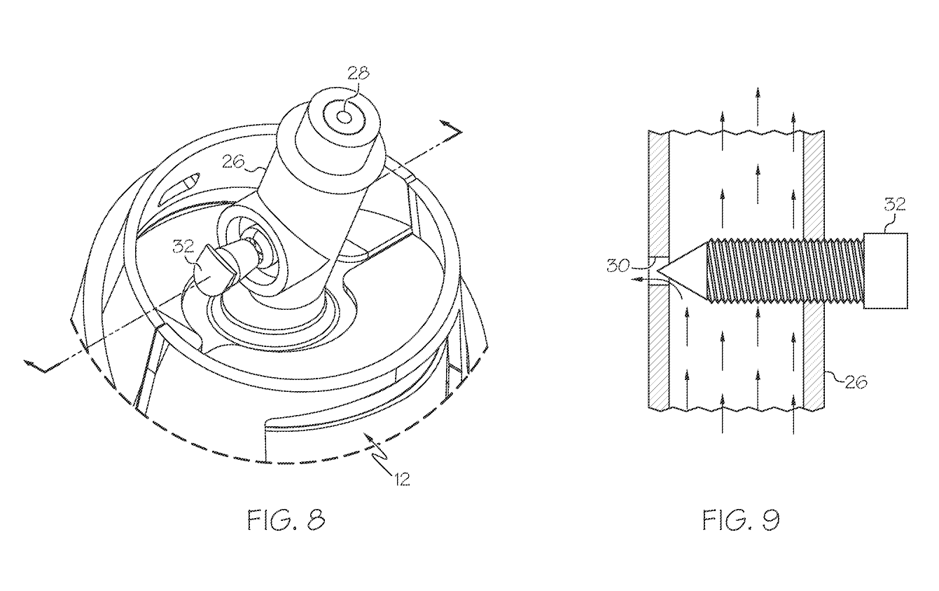

As shown in FIG. 8, when the upper end 20 is removed the air tube 26 is exposed. Air tube 26 preferably includes at least one side opening 30 which is used to regulate the pressure of air that reaches the user's nostril. As shown in FIG. 9, air flow is able to pass out of the side opening 30. However, the amount of air exiting the side opening 30 may be regulated and adjusted by the use of a screw 32 which as it is threaded further into the opening 30 will occlude more of the opening limiting air flow from the side opening 30. Likewise, backing off the screw 32 would allow more air to escape, thereby lowering the air pressure that exits nosepiece air exit opening 18.

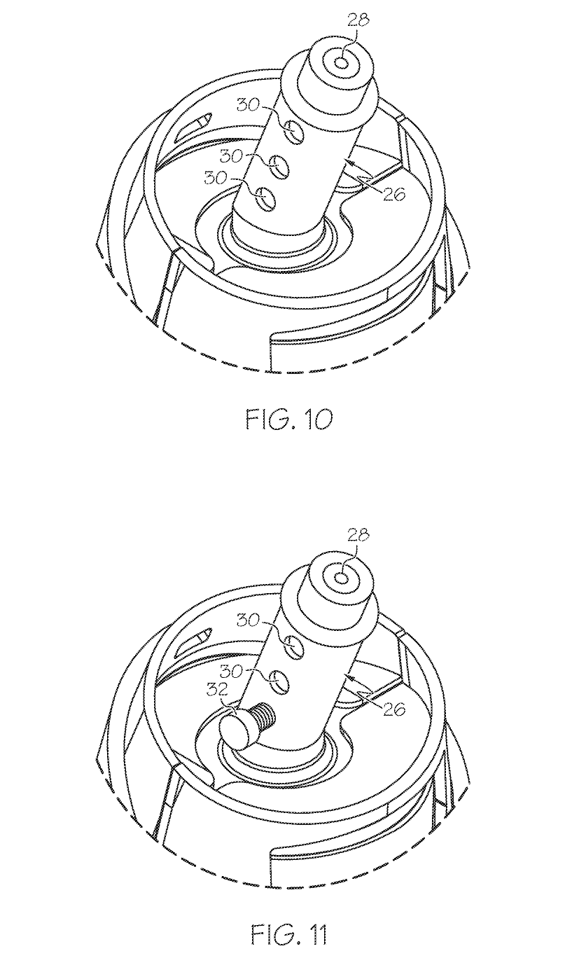

As shown in FIGS. 10 and 11, a plurality of side openings 30 may be formed in air tube 26. As in a water soaker hose, this arrangement ensures that any blockage in the nostril or sinuses will not cause a higher pressure than desired to reach the user's tissues since the plurality of openings 30 provide a mechanism to allow excess air to escape through the device without passing through nosepiece air exit opening 18. FIG. 11 shows that one or more of the side openings 30 may also include a screw 32 to be able to regulate airflow and air pressure at the factory.

It should be seen that either a plurality of side openings alone or side opening(s) with an occluding screw may be used to set the pressure of air to the nostril and to ensure that air has an exit path to prevent overpressure into the nostril. The screw 32 effectively varies the surface area of the side opening 30 to adjust the air pressure. Use of the term screw herein is intended to mean any occluding member that can alter the surface area of the side opening 30 and does not need to be a threaded screw as depicted.

It is also noted that while a battery powered air generating motor is depicted to provide pressurized air through nosepiece air exit opening 18, any mechanism that provides air under pressure may be utilized including a simple air bladder. In all cases, the air tube 26 that leads to the nosepiece air exiting opening 18 include the air pressure regulating mechanism of the invention in the form of at least one side opening with an adjustable screw. The air pressure control of the invention is very inexpensive to engineer into the device whatever the source of air under pressure.

Use of the device 10 is as in conventional air flow devices and involves placing the nosepiece(s) 16 firmly against either one nostril or both nostrils. If only a single nosepiece, then closing the other nostril such as by a finger, and switching the device on to generate air flow through air exit opening 18 into the nose. While air under pressure enters the nostril the user swallows which helps to exercise the Eustachian tubes and keep them open and pain-free.

It is believed that routine use of the device of the invention will effectively condition or exercise the sinus/Eustachian tube relationship to keep the user comfortable. Exercising the Eustachian tubes at least two times per day on a regular basis and at least two times an hour while flying may be useful in preventing ear pain. For scuba divers, repeated use of the device at least twice in the hour before diving and at least twice within an hour after diving may prevent ear discomfort. While ideal for people flying in airplanes or scuba diving where ambient pressure is changing, anyone can use the device to ensure that the Eustachian tubes are able to open easily and without pain.

When suffering from allergies, colds and any condition where the sinuses feel stuffy, ear pain may occur if the Eustachian tubes do not open as needed during swallowing and yawning. Routine use of the device of the invention several times daily may avoid ear pain and complications.

While this invention may be embodied in many different forms, there are shown in the drawings and described in detail herein specific preferred embodiments of the invention. The present disclosure is an exemplification of the principles of the invention and is not intended to limit the invention to the particular embodiments illustrated.

1. In a nasal air flow device for equalizing pressure in the Eustachian tubes including a body containing a source of air under pressure leading through an air tube to an exit port in a hand-held device, the improvement comprising:
said air tube including at least one side opening through said air tube and at least one screw mechanism inserted into at least one of said side openings to thereby function to adjust the air pressure at said exit port by altering the amount of air that can escape from said at least one side opening when said screw mechanism is positioned to occlude the side opening.

2. The nasal air flow device of claim 1 wherein said aid tube includes at least three side openings.

3. In a nasal air flow device for equalizing pressure in the Eustachian tubes including a body containing a source of air under pressure leading through an air tube to an exit port in a hand-held device, the improvement comprising:
said air tube including a plurality of side opening through said air tube sized to allow air to escape such that air pressure at said exit port is limited.

4. The nasal air flow device of claim 3 further including at least one screw mechanism inserted into at least one of said side openings to thereby function to adjust the air pressure at said exit port by altering the amount of air that can escape from said at least one side opening when said screw mechanism is positioned to occlude the side opening.

5. The nasal air flow device of claim 4 including at least three side openings.

6. A method for exercising the Eustachian tubes to limit ear pain comprising the steps of:
a) placing a nasal air flow device for exercising the Eustachian tubes with a nosepiece or nosepieces against a single nostril or both, or said nosepiece(s) including an air exit opening leading from an air tube connected to a source of air under pressure, said air tube including a plurality of side openings through said air tube sized to allow air to escape such that air pressure at said air exit opening is limited;
b) activating said nasal air flow device to release air under pressure through said air exit opening into said nostril while holding the other nostril closed;
c) swallowing while said air under pressure enters the nostril with the nasal air flow device to open the Eustachian tubes;
d) repeating use of the device at least twice daily to lessen the chance of ear pain.

7. The method of claim 6 wherein the device should be used at least two times per hour while flying in an airplane.

8. The method of claim 6 wherein the device should be used at least two times within an hour before scuba diving and at least twice within an hour after scuba diving.
